(19) United States
(12) Patent Application Publication
(10) Pub. No.: US 2011/0070342 A1
(43) Pub. Date: Mar. 24, 2011

(54) Method for evaluating and orientating baked product

(76) Inventor: Patrick J. Wilkens, Canfield, OH (US)

(21) Appl. No.: 12/807,050

(22) Filed: Aug. 26, 2010

Related U.S. Application Data
(60) Provisional application No. 61/275,141, filed on Aug. 26, 2009.

(51) Int. Cl. G01N 33/02; G05B 15/00

(52) U.S. Cl. 426/232; 700/259

(57) ABSTRACT

A method 10 for evaluating and orientating baked product includes selecting a baked product 11 that must ultimately be individually evaluated for quality, orientation and packaging in relatively large numbers. A plurality of selected baked product 11 exit an oven 9 at a relatively hot temperature and then cooled via movement on cooling conveyors 12 until being disposed upon a slicer feed conveyor 26 proximate to a vision system 14 and a robot system 16 that are supported via a support structure 18. The vision system 14 evaluates the baked product 11 for quality and orientation. The robot system 16 discards baked product 11 that does not satisfy specifications, and orientates misaligned baked product 11 that does satisfy specifications.

This Application is based on Provisional Application Ser. No. 61/275,141, filed on Aug. 26, 2009.

### BACKGROUND OF THE INVENTION

1. Field of the Invention

The present invention relates generally to a method of quality control of baked products and, more particularly, to a computerized method for evaluating and orientating baked product.

2. Background of the Prior Art

Quality control for baked products requires visually evaluating baked product parameters to determine if the baked products meet specifications. The baked product parameters can include color, length, height, undulating surfaces and surface seed quantity. Further, the baked product must be orientated correctly to make certain that the product can be slicked (if necessary) and packaged without damage.

Existing baked product computerized quality control methods provide for evaluating and removing from a conveyor belt, baked product that does not meet specifications. The existing methods do not provide for orientating the baked product (if necessary) after determining that the product meets specifications. Further, the existing methods do not provide a computerized quality control method that can be manually tuned to quickly and economically reconfigure quality control computer input and output signals to evaluate and orientate another baked product having a different size and/or configuration.

A new method is required that evaluates all baked product for quality and orientation on a moving conveyor belt, then determines which baked products must be repositioned and which baked products must be removed. Further, the new method must be capable of reconfiguring a. control computer, thereby allowing the computer to provide quality control for varying types of baked goods, including but not limited to buns, bread and donuts.

### SUMMARY OF THE INVENTION

It is a principal object of the present invention is to provide a method for evaluating and orientating baked product. A feature of the method is a programmable vision system for providing visual information about each baked product on a moving conveyor belt to a computer. An advantage of the method is that from the visual information, the computer can evaluate parameters for each baked product, including but not limited to color, length, width, surface deviations and quantity of seeds. Another advantage of the method is that the computer can determine from the evaluated parameters, if the baked product meets specifications. Yet another advantage of the method is that from the visual information, the computer can determine if each baked product is orientated pursuant to specifications.

Another object of the present invention is to provide a method for removing baked product that does not meet specifications from a conveyor belt. A feature of the method is a programmable robot that can elevate and reposition a baked product upon a moving conveyor belt, or that can remove a baked product from a moving conveyor belt. Another feature of the method is a programmable robot that can be controlled by a computer. An advantage of the method is that the computer, after evaluating baked product parameters can instruct the robot to remove baked product that does not meet specifications from the moving conveyor belt. Another advantage of the method is that the computer, after determining if a baked product is not orientated pursuant to specifications, can instruct the robot to reposition the baked product such that orientation specifications are satisfied.

Yet another object of the present invention is to allow an individual to provide algorithms to the computer that tunes the software such that the computer can evaluate and orientate, via the same vision and robot systems, a new baked product having different parameters. A feature of the method is a computer that can accept tunable software configured to provide quality control for baked products. An advantage of the method is that an entire conveyor system with quality control hardware can be reconfigured quickly and economically for a new baked product.

Briefly, the invention provides a method for evaluating and orientating a plurality of a preselected baked product, said method comprising the steps of selecting a baked product to be analyzed for quality and orientated for packaging; programming a vision system to evaluate each of a plurality of a selected baked product to determine if each one satisfies specifications; programming a robot system to discard each one of a selected baked product that does not satisfy specifications; programming a robot system to orientate each one of a selected baked product that satisfies specifications; disposing a plurality of selected baked product upon a cooling conveyor; moving the plurality of selected baked product proximate to a vision system and a robot system; determining via said vision system, if a selected one of the plurality of selected baked product satisfies specifications; removing said selected one of the selected baked product via said robot system, if said selected one does not satisfy specifications; determining if said selected one of the selected baked product that satisfies specifications is correctly orientated via said vision system; orientating said selected one of the selected baked product that satisfies specifications via said robot system, if said selected one is not correctly orientated; packaging each specification compliant and correctly orientated baked product; and determining if more baked product is required for an order placed by a customer, whereupon, if more baked product is required, then more baked product is added upon said cooling conveyor, and the added baked product is analyzed, if more baked product is not required, then the method is terminated.

The invention further provides a method for packaging a preselected baked product comprising establishing specifications for a preselected baked product; providing a vision system to evaluate each one of a preselected baked product to determine if each one of the preselected baked product is specification and orientation compliant; providing a robot system that discards each specification noncompliant preselected baked product, and that orientates each specification compliant preselected baked product that is misaligned; disposing a plurality of preselected baked product proximate to said vision system, whereupon, said vision system determines specification and orientation compliance for each one of said plurality of preselected baked product; discarding a specification noncompliant baked product via said robot system; orientating a specification compliant baked product via said robot system; and packaging each specification compliant and orientation compliant baked product.

### BRIEF DESCRIPTION OF THE DRAWINGS

These and other objects, advantages and novel features of the present invention, as well as details of an illustrative embodiment thereof, will be more fully understood from the following detailed description and attached drawings, wherein:

FIG. 1 is a flow chart for a method for evaluating and orientating baked product in accordance with the present invention.

FIG. 2 is a top elevation view of a support structure with vision and robot systems attached thereto in accordance with the present invention.

FIG. 3 is a side elevation of the of the support structure of FIG. 2.

FIG. 4 is the side elevation view of the support structure of FIG. 3, but with a robot hand included in the robot system.

FIG. 5 is a back elevation view of the support structure of FIG. 4.

FIG. 6 is a top elevation view of the support structure of FIG. 4.

FIG. 7 is a front elevation view of the support structure of FIG. 4.

FIG. 8 is a top elevation view of the support structure of FIG. 4, but with the vision and robot systems removed.

FIG. 9 is top elevation view of a support structure with a cooling conveyor for supplying baked product from a baking oven to the vision and robot systems secured to the support structure in accordance with the present invention.

FIG. 10 is a side elevation view of the support structure, cooling conveyor and baking oven of FIG. 9.

FIG. 11 is a side elevation view of a support structure with alternative vision and robot systems attached thereto in accordance with the present invention.

FIG. 12 is a top elevation view of the support structure and alternative vision and robot systems of FIG. 11.

### DESCRIPTION OF THE PREFERRED EMBODIMENT

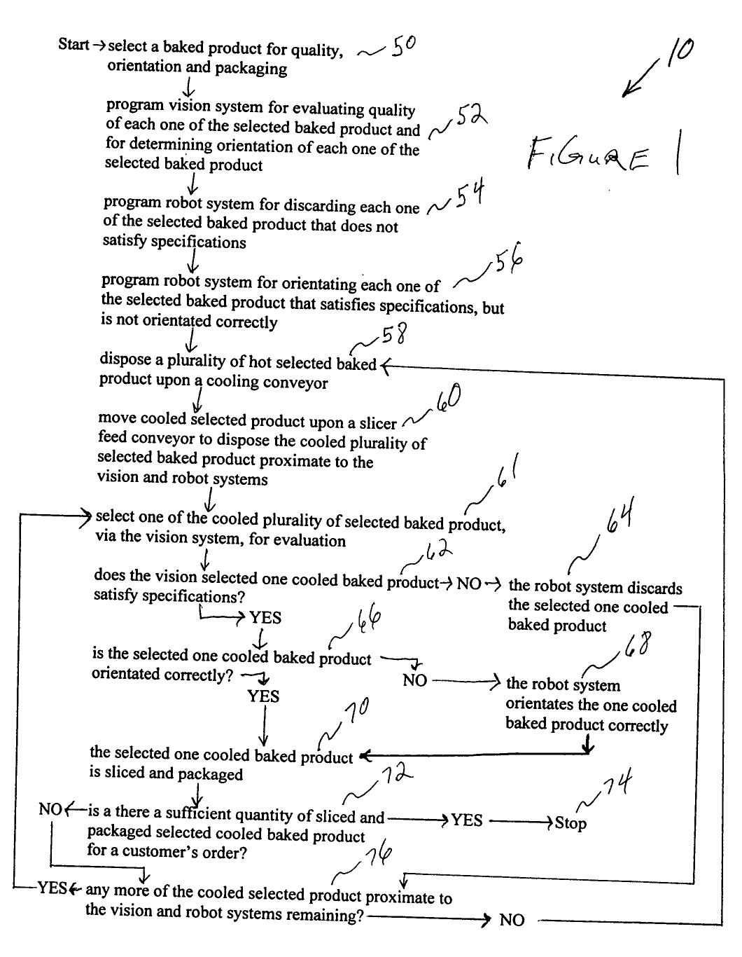

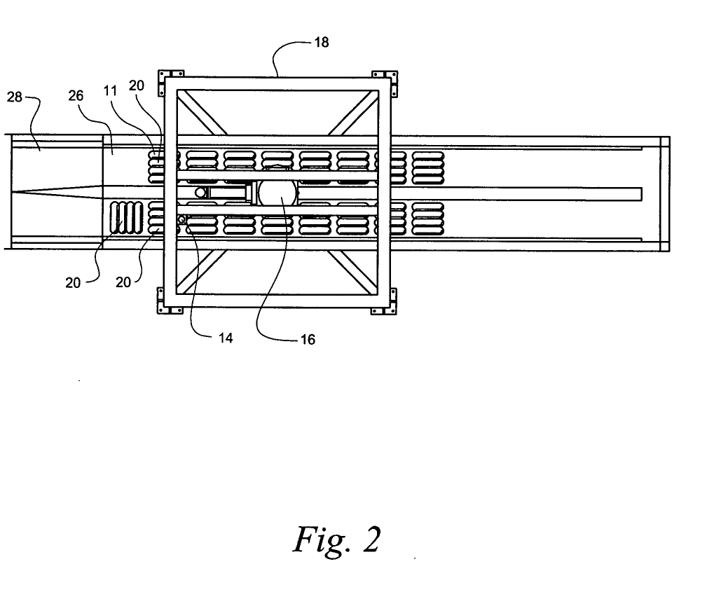

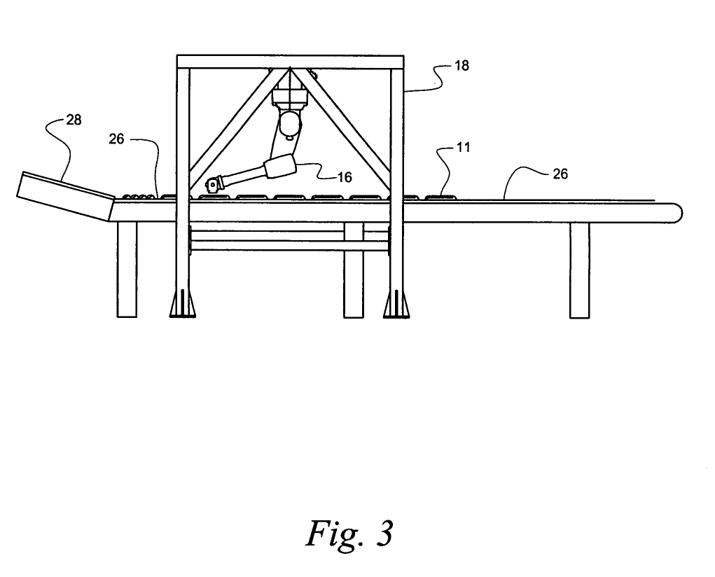

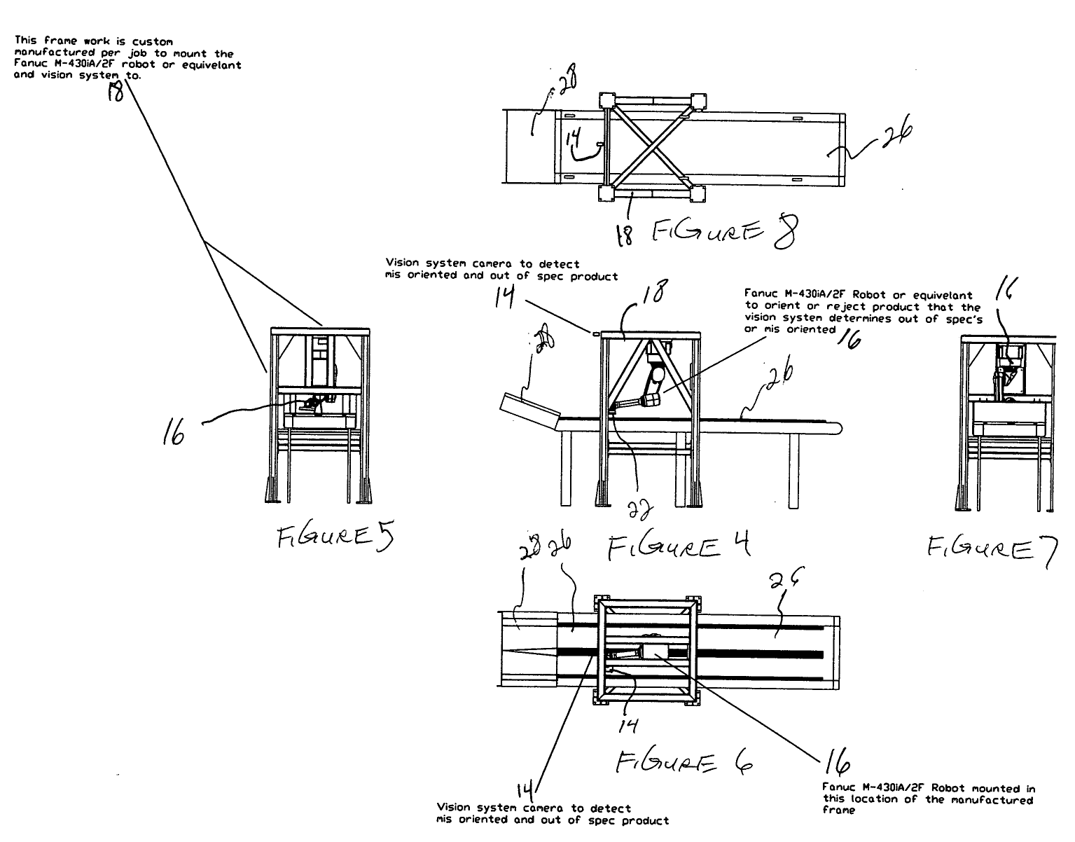

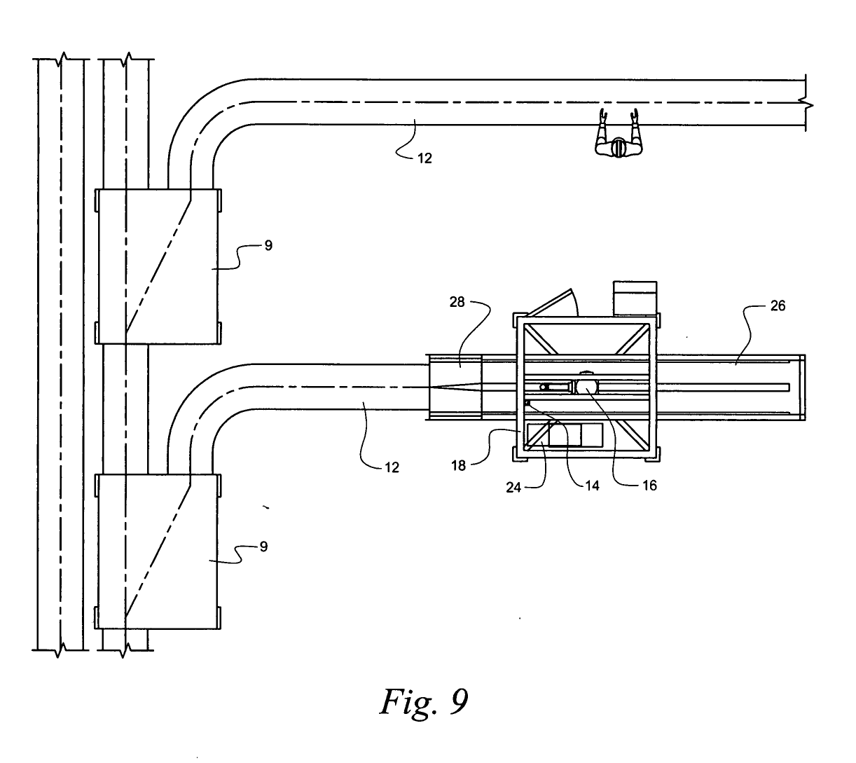

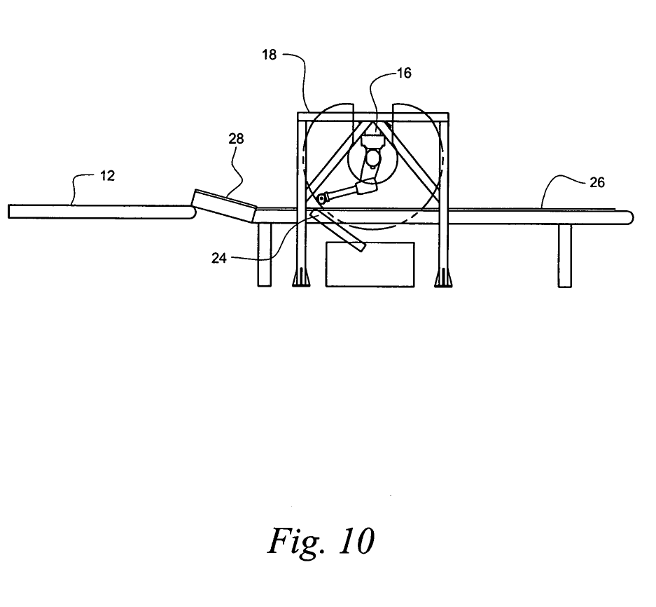

Referring now to the drawings, a method for evaluating and orientating a plurality of a preselected baked product in accordance with the invention is denoted a numeral 10. The method 10 includes selecting a baked product 11 (FIGS. 2 and 3) that must ultimately be individually evaluated for quality, orientation and packaging in relatively large numbers (FIG. 1, block 50). The baked product 11 may be selected from a myriad of products including, but not limited to buns, bread, rolls, sandwiches and dessert treats. A plurality of the selected baked product 11 ultimately exit an oven 9 (FIG. 9) at a relatively hot temperature (350-400 degrees Fahrenheit) and are then cooled to about 100-125 degrees Fahrenheit by placing the baked product upon cooling conveyors 12 (FIG. 9). As the baked product 11 is moved via the cooling conveyors 12, the baked product 11 drops to the desired 100-125 temperature range where the baked product 11 is conveyed underneath and proximate to a vision system 14 (FIGS. 2 and 9), and a robot system 16 (FIGS. 2 and 3) that are supported via a support structure 18 (FIGS. 2-10).

The vision system 14 is a model it vision system manufactured by Fanuc Corporation, which is located at Rochester Hills, Michigan. The robot system 16 is a “single arm” robot manufactured by Fanuc corporation, model number M-430iA/2F. The vision and robot systems 14 and 16 include computers interfaced and programmed to achieve the present method 10 for evaluating and orientating baked product. The support structure 18 is dimensioned and configured to dispose the vision and robot systems 14 and 16 above a predetermined portion of the cooling conveyor 12. The support structure 18 positions the vision system 14 to “see” laterally across the entire conveyor 12 to view an entire row 20 of baked product 11 to provide baked product 11 “quality control” information to the computers. The support structure 18 positions the robot system 16 to enable the robot arm to remove any baked product after being “told” to do so by the computers after the computers determine which baked products 11 do not meet specifications.

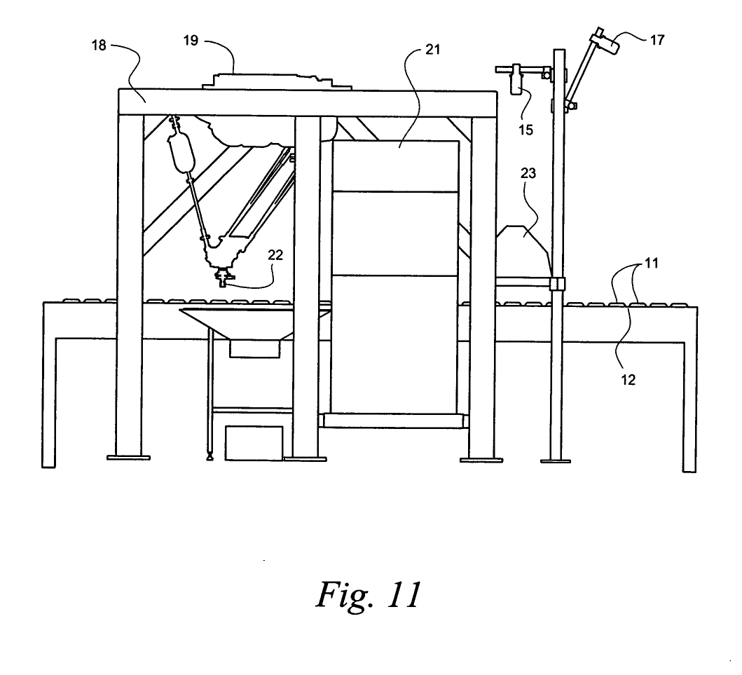

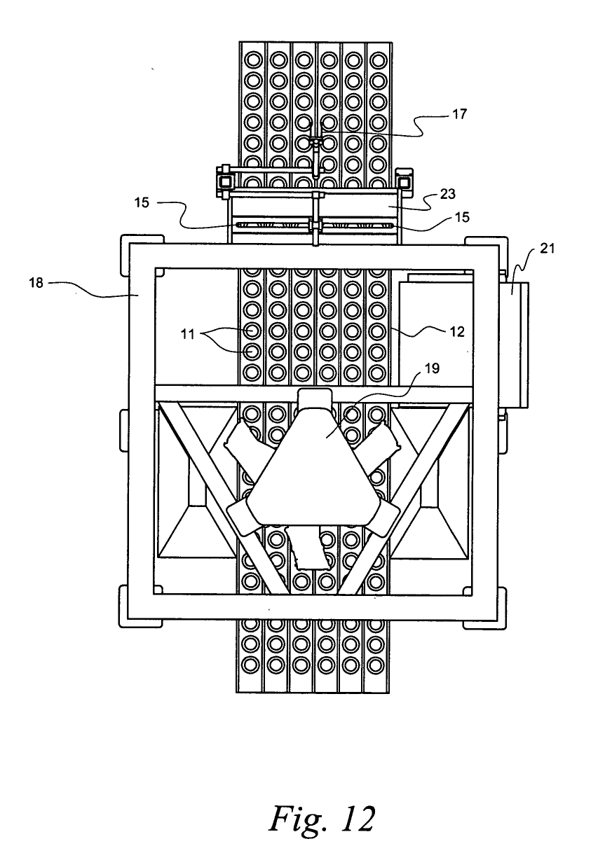

Referring to FIGS. 11-12, alternative vision and robot systems are depicted. The vision system includes a two dimensional first camera 15 that determines baked product 11 parameters including, but not limited to length, width, color, surface deviations and seed count. The vision system further includes a three dimensional second camera 17 mounted at a forty-five degree angle to the baked product 11 to determine baked product height dimensions. The first camera 15 is a Basler two dimensional camera having part number RUL209810GC and manufactured in Ahrensburg, Sweden. The second camera 17 is a Sick Ranger three dimensional camera having part number 60022152 and manufactured in Linkoping, Sweden. The first camera 15 further includes a light hood 23 having a Sick Laser (part number ILP2L11111) and white LED lighting (not depicted) under the hood 23 to eliminate ambient light to allow the first camera 15 to determine the true color of the baked product 11 passing under the first camera 15. The first and second cameras 15 and 17 provide baked product 11 quality control information to a cooperating computer 21.

The robot system includes a “spider” robot 19 manufactured by Fanuc corporation, model number M3IA. The computer 21 includes Labview software provided by National Instruments located at 11500 Mo-Pac Expy in Austin, Tex. The Labview software allows a person to program the computer 21 with algorithms that provide optimum process control for achieving a method 10 for evaluating, orientating and removing (if necessary) any baked product 11 on the conveyor 12, irrespective of configuration, including but not limited to hot dog buns, hamburger buns, bread loaves and donuts. The computer 21 ultimately instructs the robot 19, via the Labview software and cooperating algorithms, to orientate baked product 11 upon the conveyor 12, or to remove baked product 11 from the conveyor 12 that does not meet specifications. Irrespective of the vision and robot systems used to evaluate, orientate or remove baked product 11, the support structure 18 disposes the robot system 16 or 19 such that a “hand” 22 of the robot system 16 or 19 is capable of grasping individually, each one of the baked product 11 in the row 20 laterally across the conveyor 12. The hand 22 is manufactured specifically for the baked product 11 being packaged. The support structure 18 must also be dimensioned and configured to avoid any existing structures proximate to the location where the support structure 18 is to be installed. The support structure 18 is manufactured from steel and must be engineered to support the weight of the vision systems 14 or (15 and 17), and the robot systems 16 or 19 to withstand the vibrations initiated by the movement of the robot systems 16 or 19 when picking-up and orientating or discarding, via a discarding chute 24, the baked product 11.

Referring now to FIG. 1 which also applies to the alternate vision and robot systems detailed above, after selecting the baked product 11 (block 50), the vision system 14 is programmed to evaluate the quality of each one of a plurality of a selected baked product 11 to determine if each one satisfies specifications and orientation upon the cooling conveyor 12 (block 52). The specification programming includes, but is not limited to, determining the configuration and dimensions of the baked product 11, as well as color, toppings, surface blemishes, quantity of seeds and weight of the baked product 11. The orientation programming includes, but is not limited to, determining if the longitudinal and lateral orientation of the baked product 11 upon the cooling conveyor 12 is correct to provide for the consistent slicing and undamaged packaging of the baked product 11.

Referring to block 54 of FIG. 1, the robot system 16 is programmed to cooperate with the vision system 14 to ultimately discard each one of a selected baked product 11 that does not satisfy specifications. Further, the robot system 16 is programmed to cooperate with the vision system 14 to ultimately orientate each one of the selected baked product 11 that satisfies specifications, but is not orientated correctly, thereby promoting consistent slicing and undamaged packaging (block 56).

Referring to block 58, after programming the vision and robot systems 14 and 16, existing baking equipment (not part of the present invention) is energized to provide hot selected baked product 11 upon the cooling conveyor 12. The hot selected baked product 11 is ultimately cooled while being moved via the cooling conveyor 12, thereby allowing the cooling conveyor 12 to dispose the now cooled baked product 11 upon a down slide 28, which promotes the sliding of the cooled baked product 11 from the cooling conveyor 12 to and upon a slicer feed conveyor 26. The slicer feed conveyor 26 positions rows 20 of cooled baked product 11 proximate to and below the vision and robot systems 14 and 16 (block 60). The travel time for the hot selected baked product 11 to reach the slicer feed conveyor 26 is predetermined to provide a cooled baked product 11.

Referring to block 61, the vision system 14 selects one of the cooled plurality of selected baked product 11 for evaluation. The selected one baked product 11 is scanned to determine if preselected features, including but not limited to color, surface blemishes, size and presence of toppings, and quantity of seeds satisfies predetermined parameters and/or specifications (decision block 62). If the selected one cooled baked product 11 does not satisfy specifications, the robot system 16 discards the selected one baked product 11 (block 64); whereupon, the method 10 jumps to decision block 76 and determines if any more of the cooled selected product 11 proximate to the vision and robot systems 14 and 16 remains. If none of the cooled selected product 11 remains, then the method 10 jumps to block 58 where another plurality of hot selected baked product 11 is disposed upon the cooling conveyor 12 and the method 10 continues from block 58. Returning to decision block 76, if more of the cooled selected product 11 remains, then the method 10 jumps to block 61 where another one of the cooled plurality of selected baked product 11 is evaluated via the vision system 14 and the method continues from block 61.

Returning to decision block 62, if the selected one cooled baked product 11 satisfies specification, then the method 10 proceeds to decision block 66 to determine if the selected one cooled baked product 11 is orientated correctly. If the one cooled baked product 11 is not orientated correctly, then the robot system 16 orientates the one cooled baked product 11 correctly (block 68). The method 10 then proceeds to block 70 and the selected one cooled baked product 11 is moved via the slicer feed conveyor 26 to slicing and packaging means (not depicted) well known to those of ordinary skill in the art. Returning to decision block 66, if the one cooled baked product 11 is orientated correctly, then the method 10 proceeds to block 70.

After slicing and packaging the one cooled baked product 11 (block 70), the method 10 proceeds to decision block 72; whereupon, the method 10 determines if there is a sufficient quantity of sliced and packaged selected cooled baked product 11 to satisfy an order placed by a customer. If there is a sufficient quantity of sliced and packaged selected cooled baked product 11 to satisfy the customer's order, then the method 10 stops (block 74). If there is not a sufficient quantity of sliced and packaged selected cooled baked product 11, then the method 10 proceeds to decision block 76 where the method 10 determines if there remains any more of the selected cooled baked product proximate to the vision and robot systems 14 and 16. The method 10 then repeats decision block 76 as detailed above until there is a sufficient quantity of sliced and packaged selected cooled baked product 11.

1. A method for evaluating and orientating a plurality of a preselected baked product, said method comprising the steps of:
selecting a baked product to be analyzed for quality and orientated for packaging;
programming a vision system to evaluate each of a plurality of a selected baked product to determine if each one satisfies specifications;
programming a robot system to discard each one of a selected baked product that does not satisfy specifications;
programming a robot system to orientate each one of a selected baked product that satisfies specifications;
disposing a plurality of selected baked product upon a cooling conveyor;
moving the plurality of selected baked product proximate to a vision system and a robot system;
determining via said vision system, if a selected one of the plurality of selected baked product satisfies specifications;
removing said selected one of the selected baked product via said robot system, if said selected one does not satisfy specifications;
determining if said selected one of the selected baked product that satisfies specifications is correctly orientated via said vision system;
orientating said selected one of the selected baked product that satisfies specifications via said robot system, if said selected one is not correctly orientated;
packaging each specification compliant and correctly orientated baked product; and
determining if more baked product is required for an order placed by a customer, whereupon, if more baked product is required, then more baked product is added upon said cooling conveyor, and the added baked product is analyzed, if more baked product is not required, then the method is terminated.

2. The method of claim 1 wherein said step of disposing a plurality of selected baked product upon a cooling conveyor includes the step of directing baked product from a cooling conveyor onto a slicer feed conveyor.

3. The method of claim 1 wherein said step of moving the plurality of selected baked product proximate to a vision system and a robot system, includes the step of constructing framework for supporting said vision system such that said vision system is disposed for evaluating quality and determining orientation for each one of said plurality of selected baked product placed upon said slicer feed conveyor.

4. The method of claim 1 wherein said step of moving the plurality of selected baked product proximate to a visions system and a robot system, includes the step of constructing framework for supporting said robot system such that said robot system is disposed for discarding baked product that is not specification compliant and for orientating specification compliant baked products that are not correctly orientated.

5. The method of claim 1 wherein said step of determining if a selected one of the selected baked product satisfies specifications includes the step of determining via said vision system if said selected one baked product is right side up.

6. The method of claim 5 wherein said step of determining via said vision system if said selected one of the baked product is right side up includes the step of lifting and positioning the baked product, via said robot system, in a right side up position if said selected one of the baked product is not initially in a right side up position.

7. The method of claim 1 wherein said step of determining if a selected on of the selected baked product satisfies specifications includes the step of determining via said vision system if the said selected one baked product includes the correct color, size, and toppings.

8. The method of claim 1 wherein said step of removing said selected one of the selected baked product via said robot system, if said selected one does not satisfy specifications, includes the step of lifting each non-specification compliant baked product from the orientation conveyor via said robot system, and discarding each non-specification compliant baked product into a garbage bin via said robot system.

9. The method of claim 1 wherein said step of determining via said vision system if a selected one of the plurality of selected baked product satisfies specifications, includes the step of determining if each of the plurality of selected baked product satisfies specification.

10. The method of claim 1 wherein said step of removing said selected one of the selected baked product via said robot system, if said selected one does not satisfy specifications, includes the step of removing each of the selected baked product via said robot system that does not satisfy specifications.

11. The method of claim 1 wherein said step of determining if said selected one of the selected baked product that satisfies specifications is correctly orientated via said vision system, includes the step of determining if each of the selected baked product is correctly orientated.

12. The method of claim 1 wherein said step of orientating said selected one of the selected baked product that satisfies specifications via said robot system, if said selected one is not correctly orientated, includes the step of orientating each specification compliant baked product upon the conveyor via said robot system.

13. The method of claim 1 wherein said step of programming a robot system to discard each one of a selected baked product to determine if each one satisfies specifications includes a diagnostic system to determine if said robot system is within operating specifications, whereupon, said diagnostic system shuts down said robot system and all supporting systems that cooperate with said robot system.

14. A method for packaging a preselected baked product comprising:
establishing specifications for a preselected baked product;
providing a vision system to evaluate each one of a preselected baked product to determine if each one of the preselected baked product is specification and orientation compliant;
providing a robot system that discards each specification noncompliant preselected baked product, and that orientates each specification compliant preselected baked product that is misaligned;
disposing a plurality of preselected baked product proximate to said vision system, whereupon, said vision system determines specification and orientation compliance for each one of said plurality of preselected baked product;
discarding a specification noncompliant baked product via said robot system;
orientating a specification compliant baked product via said robot system; and
packaging each specification compliant and orientation compliant baked product.

15. The method of claim 14 wherein said step of providing a vision system includes the step of programming said vision system to determine color, size, and presence of toppings for the baked product.

16. The method of claim 14 wherein said step of providing a robot system includes the step of programming said robot system to discard specification noncompliant baked product.

17. The method of claim 14 wherein said step of providing a robot system includes the step of programming said robot system to orientate a specification compliant and orientation noncompliant baked product.

18. The method of claim 14 wherein said step of providing a robot system includes the step of providing a diagnostic system to determine if said robot system is within operating specifications, whereupon, said diagnostic system shuts down said robot system and all supporting systems that cooperate with said robot system if said robot system is not within operating specifications.

19. The method of claim 14 wherein said step of providing a vision system includes the step of providing an adjustable support structure that three dimensionally adjusts the position of said vision system relative to a baked product.

20. The method of claim 14 wherein said step of providing a robot system includes the step of providing an adjustable support structure that three dimensionally adjusts the position of said robot system relative to a baked product.
